(19) United States
(12) Patent Application Publication
(10) Pub. No.: US 2020/0345616 A1
(43) Pub. Date: Nov. 5, 2020

(54) PROCESS FOR STYLING HUMAN HAIR

(71) Applicant: BASF SE, Ludwigshafen (DE)

(72) Inventors: Juergen Falkowski, Düsseldorf-Holthausen (DE); Andrea Tomlinson, Cheadle (GB); Helena Wiethoff, Düsseldorf-Holthausen (DE); Nicole Kurth, Monheim (DE)

(21) Appl. No.: 16/766,765

(22) PCT Filed: Nov. 19, 2018
(86) PCT No.: PCT/EP2018/081677

§ 371 Date: May 26, 2020

(30) Foreign Application Priority Data
Nov. 29, 2017 (EP) 17204291.3

(51) Int. Cl. A61K 8/81; A61Q 5/06; A61Q 5/04; A45D 7/06

(52) U.S. Cl. CPC A61K 8/8188; A61Q 5/06; A61K 2800/594; A45D 7/06; A61Q 5/04

(57) ABSTRACT

The present invention relates to a composition comprising a polymer, wherein this polymer is water-soluble and has a melting point of at least 145° C. and comprises sulfonic acid groups, and an organic amine having at least two amino groups, and optionally further cosmetic ingredients. Furthermore, the present invention relates to a process for restyling human hair comprising applying to the human hair a composition comprising a polymer, wherein this polymer is water-soluble and has a melting point of at least 145° C. and comprises sulfonic acid groups, and an organic amine, and optionally further cosmetic ingredients, thereafter heat-treating the hair, together with the composition on its surface, thereafter allowing the hair to cool down to a temperature of below 100° C., and thereafter changing the hair style by heat-treating the hair. Furthermore, the present invention relates to the use of the said polymer or of the said composition for restyling human hair.

The present invention relates to a composition comprising a polymer, wherein this polymer is water-soluble and has a melting point of at least 145° C. and comprises sulfonic acid groups, and an organic amine having at least two amino groups, and optionally further cosmetic ingredients. Furthermore, the present invention relates to a process for restyling human hair comprising applying to the human hair a composition comprising a polymer, wherein this polymer is water-soluble and has a melting point of at least 145° C. and comprises sulfonic acid groups, and an organic amine, and optionally further cosmetic ingredients, thereafter heat-treating the hair, together with the composition on its surface, thereafter allowing the hair to cool down to a temperature of below 100° C., and thereafter changing the hair style by heat-treating the hair. Furthermore, the present invention relates to the use of the said polymer or of the said composition for restyling human hair.

Hair Styling products containing water or ethanol soluble polymers are very popular products in today's cosmetic market. Such products typically involve a liquid or wax/gel-like composition which is applied on wet or dry hair to obtain a certain long-lasting hair style. They can be produced as aerosol sprays, sprays, mousses, gels, waxes, serums or other textures. Apart from the polymer respectively the polymer combination a large number of ancillary ingredients is used to adjust certain texture or performance effects.

The most important performance of hair styling products is the stiffness respectively hold of the hair style and the ability to resist high humidity conditions. Natural hair can also be styled by using blow dryers or straightening/curling devices. The most important disadvantages of only heat-treated hair are the low longevity of the hair style and the sensitivity against moisture. Similar to synthetic polymers natural hair also has a glass transition temperature (T_{g}) depending on its water content. The T_{g} of absolute dry human hair (0% water content) is around 144° C. It decreases with increasing water content and reaches about 0° C. at about 23% water content.

The humidity condition to obtain very low water content (<5 wt.-%) of hair can almost be achieved during hair straightening with heated devices. Straightening irons therefore should be operated at least at 150° C. for styling hair. The relative humidity within buildings is typically between 40% RH and 60% RH. Depending on season and weather conditions the relative humidity outside can be between 20% RH and 90% RH. As a consequence, natural hair takes up water and the T_{g} decreases below room temperature or ambient temperature. If the hair has only been blow-dried or heat-treated the hair style is lost and a new hair styling procedure is necessary. Hair styling products containing humidity resistant polymers avoid this drawback of natural hair.

Devices like straightening irons nevertheless are very popular because they can—depending on consumer preference—either straighten curly hair or curl straight hair. As mentioned before a temperature for these devices of at least 150° C. is necessary to form hair to another style. Many straightening irons are using even temperatures of up to 230° C. to boost or shorten the styling procedure. The major drawback of these high-temperature treatments is the irreversible damage of the hair at temperatures beyond about 180° C. and especially beyond about 200° C. There are some products in the hair styling market claiming heat-protection in this temperature range. These products are forming films around the hair fibers and try to prevent a direct contact of the heated the iron with outer hair surface called cuticle. Nevertheless—depending on consumer habits—they cannot prevent hair damages completely. Many straightening irons or crimpers are therefore nowadays operating at lower temperatures, like 170° C.-185° C. to prevent hair damage.

Most synthetic polymers have a glass transition temperature T_{g}. There are standard polymers like PP which are continuously produced using special catalysts to adjust molecular weight distribution and thus the melting point and T_{g}. These polymers can be melted easily due to their narrow molecular weight distribution and they are for example used in the production of synthetic fibers/fabrics in a melt process. Polymers which are typically used in the cosmetic industry should in general be water soluble or ethanol soluble. They are typically produced in batch processes and most of them comprise special monomers to adjust parameters like solubility, water resistance or sensorial properties. They also have measurable glass transition temperatures but their melting behavior is different from polymers like PP and normally there is no real liquid/low viscous phase transition and some of them even decompose at higher temperatures. If these polymers are applied on hair and treated at high temperatures like 200° C. they are often sticky and highly viscous. These physical properties have a negative impact on the application. The sliding or gliding of straightening irons is hampered and in some cases the residence time of the heated iron on hair is increased resulting in further hair damage.

It is desirable that hair styling products allow easy restyling of hair. Restyling means that after a first hair style has been made, and has been worn for some time, the hair style is changed, e. g. from curly to flat or vice versa.

The problem underlying the present invention is to provide a process for restyling human hair which results in low or no damage to the hair, i. e. which operates at moderate temperatures, and which results in a styling effect that is more stable than simply heat treated natural hair. Furthermore, the problem underlying the present invention is to provide compositions for restyling human hair.

This problem is solved by the composition according to the present invention and by the process according to the present invention. This composition is one subject of the present invention. This process is another subject of the present invention.

The composition according to the present invention A composition comprising a polymer, wherein this polymer is water-soluble and has a melting point of at least 145° C., preferably at least 160° C., and comprises sulfonic acid groups, and an organic amine having at least two amino groups, and optionally further cosmetic ingredients, preferably selected from the group consisting of solvents, rheology modifiers, preservatives, surfactants, emulsifiers, perfumes, pigments and coloring agents.

The process according to the present invention is a process for restyling human hair comprising applying to the human hair a composition comprising a polymer as defined in the previous paragraph and an organic amine, preferably selected from the group consisting of monoethanolamine, diethanolamine, triethanolamine, aminomethyl propanol, ethylendiamine, diethylenetriamine, triethylenediamine, tetraethylenepentamine and tetrahydroxypropyl ethylenediamine, preferably in an amount as defined in claim 6, and optionally further cosmetic ingredients, thereafter heat-treating the hair, together with the composition on its surface, at a temperature of 145 to 250° C., preferably 150 to 230° C., more preferably 150 to 190° C., more preferably 160 to 185° C., wherein the hair-style is changed during this heat treatment, thereafter allowing the hair to cool down to a temperature of below 100° C., and thereafter changing the hair style by heat-treating the hair, together with the composition on its surface, at a temperature of 145 to 250° C., preferably 150 to 230° C., more preferably 150 to 190° C., more preferably 160 to 185° C.

Water-soluble means that at 20° C. it is possible to make a solution of at least 0.1% polymer in water, preferably at least 2% polymer in water (without adding neutralizing agents or other additives).

The melting point of the polymer can be determined visually. The polymer in powder form is filled in a thin glass tube and heated until the initially non-transparent powder becomes clear, i. e. becomes a melt. The heating rate is chosen low so that further lowering of the heating rate does not result in determining a lower melting point.

Tetrahydroxypropyl ethylenediamine can be commercially obtained from BASF (the trademark of BASF is Neutrol® TE from BASF)

In one embodiment of the composition according to the present invention or of the process according to the present invention a formulation is used and the formulation comprises at least one further polymer, wherein this further polymer is used preferably at a concentration of 0.1 to 10% by weight.

In one embodiment of the composition according to the present invention or of the process according to the present invention a formulation is used and the formulation comprises at least one further polymer, wherein this further polymer is cationic and used preferably at a concentration of 0.1 to 10% by weight.

In one embodiment of the composition according to the present invention or of the process according to the present invention a formulation is used and the formulation comprises at least one further polymer, wherein this further polymer is anionic and used preferably at a concentration of 0.1 to 10% by weight.

In one embodiment of the composition according to the present invention or of the process according to the present invention a formulation is used and the formulation comprises at least one further polymer, wherein this further polymer is nonionic and used preferably at a concentration of 0.1 to 10% by weight.

In one embodiment of the composition according to the present invention or of the process according to the present invention a formulation is used and the formulation comprises a combination of different polymers, wherein these further polymers are used together at a total concentration of 0.1 to 15% by weight, preferably at a total concentration of 0.1 to 10% by weight.

The combination with a cationic polymer in general leads to a higher stiffness and to a better hold of the hair style, curl retention and weather resistance are improved by the presence of the cationic polymer.

In one embodiment of the present invention the polymer in the composition according to the present invention is selected from the group consisting of polyacrylamidomethylpropane sulfonic acid and polystyrene sulfonic acid, preferably the polymer is polyacrylamidomethylpropane sulfonic acid.

The described organic neutralizing agents can also be combined so that a mixture of inorganic and organic neutralizing agents is used or using a mixture of different organic neutralizing agents is used in an amount that leads to partial or complete neutralization of the polymer, so that the composition preferably has a pH value of 1 to 14, preferably 3 to 9.

Surprisingly it has been found that the melting behavior and the styling effect after heat activation of the preferred polymer can be adjusted by the choice of the neutralizing agent. Example 6 describes this surprising effect by measuring bending stiffness values of heat treated hair strands. The use of different neutralizing agents results in significantly different stiffness values after heat treatment. Organic neutralizing agents yield to high stiffness values presumably because the heat transfer from the straightening iron can probably melt the polymer on the hair strand and thus reconnect the hair fibers after cooling. This surprising process does not work in formulations containing polyacrylamidomethylpropane sulfonic acid neutralized with sodium hydroxide, but is most effective (highest stiffness value) with the organic neutralizing agent Neutrol® TE from BASF (tetrahydroxypropyl ethylenediamine). There is no exact scientific explanation for the different physicochemical phase behavior of the differently neutralized polymers but it can be favorably used to tweak formulations with respect to stiffness or suppleness. It is assumed that the presence of more than one amino group in the amine used to neutralize the polymer contributes to the superior properties of the compositions comprising tetrahydroxypropyl ethylenediamine.

Example 7 shows that the bending stiffness values of only dried polymer solutions are all on the same level and practically independent from the neutralizing agent. The absolute bending stiffness values are higher than in Example 6 because the dried strands have a larger cross-sectional area when they were broken by the cantilever of the test instrument.

Hair strands treated with differently neutralized agents of the preferred polymer can also be used to restyle the hair with different styling devices, like hair straighteners, crimp irons or curling irons in the preferred temperature range from 150° C. to 190° C. Example 8, 9 and 10 show this surprising effect. In compliance with the bending stiffness values from example 6 best restyling effects can be obtained when Neutrol® TE is used as a neutralizing agent. Other organic amines, like triethanolamine or aminomethyl propanol also lead to good results. Examples 11 and 12 compare this surprising effect with formulations containing different neutralizing agents. It can be clearly seen on the images (figures) that the hair strands treated with the inorganic neutralizing agent have spread, best styling results can be achieved with Neutrol® TE. The restyling procedure can also be repeated with different styling devices because the melting of the polymer is a reversible process. The hair itself is not damaged during these restyling procedures because of the moderate temperature range from 150° C. to 190° C. and the choice of the neutralizing agent allows the adjustment of the stiffness of the hair style, like curly or straight hair.

As described above the choice of the neutralizing agent influences the stiffness of the heat-treated hair strands. On the images (figures) of Examples 11 and 12 it could also be shown that there is less frizz on hair strands treated and heat activated with the preferred neutralizing agents and the polymer polyacrylamidomethylpropane sulfonic acid. Especially hair strands showing higher stiffness values are almost free from frizz after heat activation in the preferred temperature range from 150° C. to 190° C. This is also a main advantage compared to only water-treated strands which are styled in this temperature range.

Example 13 shows that the anti-frizz effect of a formulation containing Neutrol® TE works also when the hair strand had been exposed to high humidity.

In one embodiment of the present invention the composition is a rinse-off composition selected from the group consisting of a hair shampoo and a shower gel, each containing anionic surfactants or betaine surfactants or nonionic surfactants.

In one embodiment of the present invention the composition is a conditioner containing at least one anionic, nonionic or cationic surfactant.

Another subject of the present invention is the use of a polymer according to the present invention, or of a formulation or composition as defined before for styling human hair. Preferably this use is carried out in a process according to the present invention.

In one embodiment of the process according to the present invention the formulation used in this process is selected from the group consisting of a gel, a mousse, a spray, a serum, a cream and a wax.

In one embodiment of the composition according to the present invention the composition is a non-aerosol composition, and the composition is selected from the group consisting of a gel, a mousse, a spray, a serum, a cream and a wax.

The polymer according to the present invention is water-soluble and thus can be easily washed out of the hair.

The polymer according to the present invention can be “heat-activated” by melting it. This polymer can be combined with other cationic, anionic or nonionic polymers, preferably in non-aerosol formulations.

The polymer according to the present invention can be used in formulations, preferably non-aerosol formulations, containing at least one further, cationic polymer, e. g. Polyquaternium-4, Polyquaternium-11, Polyquaternium-16, Polyquaternium-46 or Polyquaternium-68.

The polymer according to the present invention can be used in formulations, preferably non-aerosol formulations, containing at least one further, anionic polymer, e. g. Acrylates Copolymers, Acrylate/Methacrylamide Copolymers, Acrylates/t-Butylacrylamide Copolymers, Vinylacetate/Crotonates Copolymers, Vinylacetate/Crotonates/Vinyl Neodecanoate Copolymers or Polyurethanes. In can also be used in formulations further comprising at least one further nonionic polymer, e. g. polyvinylpyrrolidon, Polyvinylpyrrolidon/Vinylacetate Copolymers, Polyvinylcaprolactams or Polyvinylpyrrolidon/Methacrylamide/Vinylimidazole Copolymers.

The formulations or compositions according to the present invention can comprise solvents, e. g. ethanol or propanol. They can also comprise other ancillary ingredients like glycerin, sorbitol, panthenol, proteins, rheology modifiers, preservatives, surfactants, emulsifiers, perfumes, pigments and/or coloring agents.

The polymer according to the invention can be formulated in various hair care and styling formulations. Due to its anionic nature it can also be formulated in rinse-off formulations, e. g. in hair shampoos containing ether sulfate surfactants, like laureth sulfate, and/or betaine surfactants, like cocoamidopropyl betaine, or nonionic surfactants, like alkyl polyglucosides or ethoxylated fatty alcohols. The use in leave-on conditioners for hair care containing cationic surfactants is possible and can further enhance wet and dry combing.

A possible application procedure of the polymer according to the present invention is as follows. After the product (polymer or formulation or composition comprising the polymer) has been applied on wet hair using e.g. a spray, hair cream, serum or conditioner, the hair is dried by using a blow-dryer and a combing device, like brushes or combs. The polymer facilitates the blow-drying/combing procedure and the polymer is homogenously distributed on the hair surface. Afterwards the applied polymer can be “heat-activated” using commercially available styling devices like straightening irons, curling irons or crimpers.

Due to the lubricity of the polymer according to the present invention the application of the styling devices is smooth-running compared to other polymers which do not change their phase in the preferred temperature range from 150° C. to 190° C. The sliding or gliding of the styling devices is not hampered and the residence time of the heated iron on hair is further reduced. That is why possible damages of the hair are further diminished.

If desired it is also possible to restyle the hair, like curling of straightened hair, whilst the polymer film is on the hair. The polymer film can also be easily washed out using standard hair shampoos.

All in all the composition according to the present invention offers a lot of new features for hair styling and hair care formulations to prevent hair damages due to styling or restyling with heat treatment devices.

The polymer according to the present invention can be applied to surfaces like human hair or natural respectively artificial fibers. They are especially useful for hair styling products like gels, creams, mousses serums or sprays to prevent hair damage during procedures like hair straightening or hair curling at elevated temperatures.

Further cosmetic ingredients suitable for use in the compositions according to the present invention have been described in the previous paragraphs. Furthermore, the ingredients used in the examples of the present text are suitable. Furthermore, there are numerous textbooks known to the person skilled in the art listing cosmetic ingredients suitable for use in the compositions according to the present, e. g. the “International Cosmetic Ingredient Dictionary and Handbook”, 16^{th} edition, 2016, edited by the CTFA.

### EXAMPLES

Concentrations are given in weight-% (wt %) unless another unit is given explicitly.

Concentrations mean concentrations of active matter (AM), unless another definition is given explicitly.

Rheocare® HSP 1180 from BASF which is used for the following examples and formulations is a product that has a polymer content of between 14 wt.-% AM and 18 wt.-% AM (active matter). Thus, the use of 12.5 wt.-% Rheocare® HSP 1180 in a formulation means 2 wt.-% AM polymer in the resulting formulation.

### Example 1

Following polymer solutions were prepared:
Polyacrylamidomethylpropane Sulfonic Acid (Rheocare® HSP 1180 from BASF), 1 wt.-% active matter, adjusted to pH=6 by aminomethyl propanol
Polyacrylamidomethylpropane Sulfonic Acid (Rheocare® HSP 1180 from BASF), 2 wt.-% active matter, adjusted to pH=6 by aminomethyl propanol
Polyacrylamidomethylpropane Sulfonic Acid (Rheocare® HSP 1180 from BASF), 2 wt.-% active matter, adjusted to pH=4 by aminomethyl propanol
Polyacrylamidomethylpropane Sulfonic Acid (Rheocare® HSP 1180 from BASF), 5 wt.-% active matter, adjusted to pH=6 by aminomethyl propanol

In each experiment one hair strand (Caucasian hair, 2 g/15 cm length, round-shaped) was treated with these different polymer solutions by dipping twice into the solution. The redundant product was removed by squeezing with fingers. Then the wet hair strand was dried and combed-out. Then bending stiffness after dry combing and after heat treatment using a straightening iron (supplier: Remington; Sleek and Curl, S1051) at 150° C. respectively 160° C. was determined using a 3-point method (this was done with a T.A.Plus Texture Analyzer; supplier: Stable Micro Systems) in a climatized chamber at 21° C. and 65% relative humidity. The following values were obtained:

| | Bending | Bending Stiffness | Bending Stiffness |
|---|---|---|---|
| | Stiffness after | after heat treatment | after heat treatment |
| | dry combing | at 150° C. | at 160° C. |
| 1 wt.-%, pH = 6 | 14 cN | 16 cN | 22 cN |
| 2 wt.-%, pH = 6 | 13 cN | 30 cN | 56 cN |
| 2 wt.-%, pH = 4 | 13 cN | 27 cN | 87 cN |
| 5 wt.-%, pH = 6 | 20 cN | 88 cN | 88 cN |

The higher the value of the bending stiffness the more effective and stable is the styling of the hair.

### Example 2

The following polymer solutions diluted to 2 wt.-% active matter and adjusted to pH=6 were tested:
Polyacrylamidomethylpropane Sulfonic Acid (Rheocare® HSP 1180 from BASF)
Acrylates Copolymer (Luviflex® Soft from BASF)
VP/Methacrylamide/Vinyl Imidazole Copolymer (Luviset® Clear from BASF)

In each case 10 hair strands (Caucasian hair, 2 g/15 cm length, round-shaped) were treated with these different polymer solutions by dipping twice into the solution. The redundant product was removed by squeezing with fingers. Then the wet hair strands were dried by hanging vertically in a climatized chamber at 21° C. and 65% relative humidity (RH). Finally the bending stiffness after drying, after dry combing and after heat treatment using a straightening iron at 160° C. was determined using a 3-point method (T.A.Plus Texture Analyzer; supplier: Stable Micro Systems). The following values were obtained:

| | Bending Stiffness | Bending Stiffness | Bending Stiffness |
|---|---|---|---|
| | after drying | after dry combing | after heat treatment |
| Rheocare ® | 573 +/− 76 cN | 34 +/− 12 cN | 63 +/− 2 cN |
| HSP 1180 | | | |
| Luviflex Soft | 390 +/− 40 cN | 29 +/− 5 cN | 25 +/− 5 cN |
| Luviset Clear | 674 +/− 90 cN | 28 +/− 3 cN | 32 +/− 8 cN |

Only Rheocare® HSP 1180 resulted in a significant increase of bending stiffness after heat treatment.

### Example 3

10 hair strands (Caucasian hair, 2 g/15 cm length, round-shaped) were dipped twice into a 2 wt.-% solution of polyacrylamidomethylpropane sulfonic acid which had been adjusted to pH=6 by aminomethyl propanol. The redundant product was removed by squeezing with fingers. Then the wet hair strands were blow-dried using a commercially available blow dryer and simultaneously combed during the blow drying procedure. Finally the bending stiffness after blow drying/combing and after heat treatment using a straightening iron at 160° C. was determined in a climatized chamber at 21° C. and/65% RH using a 3-point method (T.A.Plus Texture Analyzer; supplier: Stable Micro Systems). The following values were obtained:

| | After Blow Drying/Combing | After Heat Treatment |
|---|---|---|
| Bending Stiffness | 32 +/− 6 cN | 259 +/− 52 cN |

It can be concluded that combing during blow drying improves the distribution of the polymer and thus results in a higher increase of the bending stiffness after heat treatment.

### Example 4

10 hair strands (Caucasian hair, 2 g/15 cm length, round-shaped) were dipped twice into a 2 wt.-% solution of polyacrylamidomethylpropane sulfonic acid which had been adjusted to pH=1.5 by aminomethyl propanol. The redundant product was removed by squeezing with fingers. Then the wet hair strands were blow-dried using a commercially available blow dryer and simultaneously combed during the blow drying procedure. Finally the bending stiffness after blow drying/combing and after heat treatment using a straightening iron at 160° C. was determined in a climatized chamber at 21° C. and 65% RH using a 3-point method (T.A.Plus Texture Analyzer; supplier: Stable Micro Systems). The following values were obtained:

| | After Blow Drying/Combing | After Heat Treatment |
|---|---|---|
| Bending Stiffness | 35 +/− 8 cN | 216 +/− 34 cN |

The comparison of examples 3 and 4 shows that pH 1.5 and 6 lead to comparable results.

### Example 5

The following freeze-dried samples of polymer solutions were compared according to ISO 6321 (e.
polyacrylamidomethylpropane Sulfonic Acid (Rheocare® HSP 1180 from BASF), adjusted to pH=6 by aminomethyl propanol
vinylpyrrolidon/vinylacetate-copolymer (Luviskol® VA 64 from BASF)

It was found that polyacrylamidomethylpropane Sulfonic Acid (Rheocare® HSP 1180 from BASF) has a melting point at about 160° C., whereas vinylpyrrolidon/vinylacetate-copolymer (Luviskol® VA 64 from BASF) decomposes at about 260° C. without melting.

### Example 6

Following formulations with different neutralizing agents (composition see example formulations) were prepared:
Formulation no. HB-DE-16-121-001 (neutralized with sodium hydroxide)
Formulation no. HB-DE-16-121-008 (neutralized with sodium hydroxide)
Formulation no. HB-DE-16-121-012 (neutralized with aminomethyl propanol)
Formulation no. HB-DE-16-121-013 (neutralized with Neutrol® TE)
Formulation no. HB-DE-16-121-018 (neutralized with aminomethyl propanol)
Formulation no. HB-DE-16-121-019 (neutralized with sodium hydroxide)
Formulation no. HB-DE-16-121-020 (neutralized with aminomethyl propanol)
Formulation no. HB-DE-16-121-021 (neutralized with triethanolamine)

In each experiment 10 hair strands (Caucasian hair, 2 g/15 cm length, round-shaped) were treated with these different formulations by dipping twice into the solution. The redundant product was removed by squeezing with fingers. Then the wet hair strands were dried and combed-out. After heat treatment using a straightening iron at 160° C. the bending stiffness of the flattened hair strands were determined using a 3-point method (this was done with a T.A.Plus Texture Analyzer; supplier: Stable Micro Systems) in a climatized chamber at 21° C. and 65% relative humidity. The following values were obtained:

| | Bending Stiffness after heat |
|---|---|
| Formulation no. | treatment at 160° C. [cN] |
| only water | 24.1 +/− 6.8 |
| HB-DE-16-121-001 | 16.1 +/− 1.5 |
| HB-DE-16-121-008 | 17.3 +/− 1.3 |
| HB-DE-16-121-012 | 106.2 +/− 13.3 |
| HB-DE-16-121-013 | 192.4 +/− 32.1 |
| HB-DE-16-121-018 | 21.9 +/− 3.9 |
| HB-DE-16-121-019 | 64.1 +/− 27.2 |
| HB-DE-16-121-020 | 101.4 +/− 7.3 |
| HB-DE-16-121-021 | 97.09 +/− 8.9 |

The formulations HB-DE-16-121-001, HB-DE-16-121-013, HB-DE-16-121-020 and HB-DE-16-121-021 had the same chassis, only the neutralizing agents had been changed:

| | Neutralizing | Bending Stiffness after heat |
|---|---|---|
| Formulation no. | Agent | treatment at 160° C. [cN] |
| HB-DE-16-121-001 | Sodium Hydroxide | 16.1 +/− 1.5 |
| HB-DE-16-121-013 | Neutrol TE | 192.4 +/− 32.1 |
| HB-DE-16-121-020 | Aminomethyl | 101.4 +/− 7.3 |
| | propanol | |
| HB-DE-16-121-021 | Triethanolamine | 97.09 +/− 8.9 |

### Example 7

Following formulations with same chassis, but different neutralizing agents (composition see example formulations) were prepared:
Formulation no. HB-DE-16-121-001 (neutralized with sodium hydroxide)
Formulation no. HB-DE-16-121-013 (neutralized with Neutrol® TE)
Formulation no. HB-DE-16-121-020 (neutralized with aminomethyl propanol)
Formulation no. HB-DE-16-121-021 (neutralized with triethanolamine)

In each experiment 10 hair strands (Caucasian hair, 2 g/15 cm length, round-shaped) were treated with these different formulations by dipping twice into the solution. The redundant product was removed by squeezing with fingers. Then the wet hair strands were only dried overnight in a climatized chamberer at 21° C. and 65% relative humidity. Then bending stiffness of the round-shaped and dry strands were determined using a 3-point method (this was done with a T.A.Plus Texture Analyzer; supplier: Stable Micro Systems) in a climatized chamber at 21° C. and 65% relative humidity. The following values were obtained:

| | | Bending Stiffness after |
|---|---|---|
| Formulation no. | Neutralizing Agent | Drying at 21° C. [cN] |
| HB-DE-16-121-001 | Sodium Hydroxide | 465.5 +/− 48.8 |
| HB-DE-16-121-013 | Neutrol TE | 476.2 +/− 24.8 |
| HB-DE-16-121-020 | Aminomethyl | 484.7 +/− 44.7 |
| | propanol | |
| HB-DE-16-121-021 | Triethanolamine | 399.4 +/− 42.1 |

### Example 8

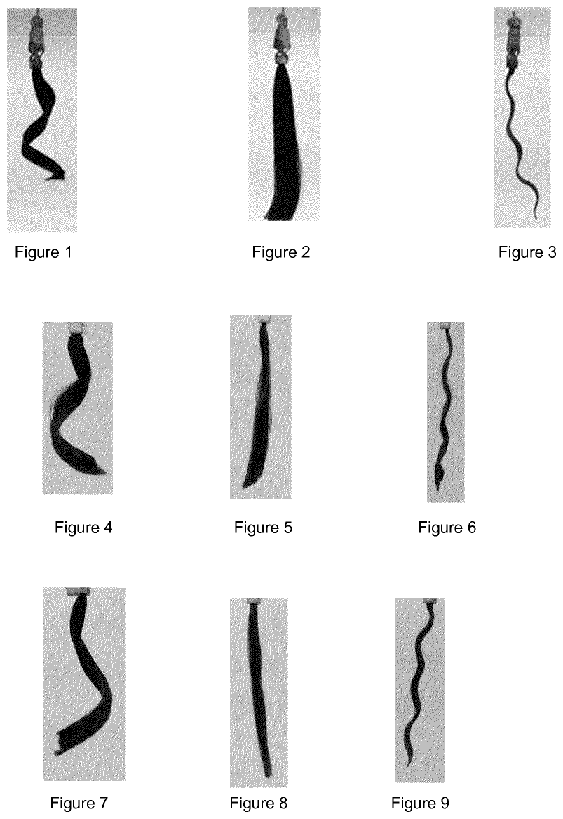

One hair strand treated with formulation no. HB-DE-16-121-013 from example 6 which had been activated by a straightening iron at 160° C. was restyled using a Babyliss Pro from Miracurl to get a curly strand (FIG. 1, left). Afterwards the same strand was treated again using a straightening iron (Braun Satin Hair 7) to get straight hair (FIG. 2, middle). Finally, the strand was treated using a curling iron (BaByliss Pro fashion attitude) to get definitely curled hair (FIG. 3, right).

The procedures can be continued with other styling devices adjusted to the preferred temperature range from 160° C.-185° C. without damaging the hair. If desired the product can be washed out.

It is also possible to spray e.g. formulation no. HB-DE-16-121-013 onto single hair strands on the head to create special hair style effects with better hold and humidity resistance than only water-treated strands (see example 8).

### Example 9

One hair strand treated with formulation no. HB-DE-16-121-020 from example 6 which had been activated by a straightening iron at 160° C. were restyled using a Babyliss Pro from Miracurl to get a curly strand (FIG. 4, left).

Afterwards the same strands were treated again using a straightening iron (Braun Satin Hair 7) to get straight hair (FIG. 5, middle). Finally, the strand was treated using a curling iron (BaByliss Pro fashion attitude) to get definitely curled hair (FIG. 6, right).

### Example 10

One hair strand treated with formulation no. HB-DE-16-121-021 from example 6 which had been activated by a straightening iron at 160° C. were restyled using a Babyliss Pro from Miracurl to get a curly strand (FIG. 7, left).

Afterwards the same strands were treated again using a straightening iron (Braun Satin Hair 7) to get straight hair (FIG. 8, middle). Finally, the strand was treated using a curling iron (BaByliss Pro fashion attitude) to get definitely curled hair (FIG. 9, right).

### Example 11

Four hair strands treated with formulation no. HB-DE-16-121-001, HB-DE-16-121-013, HB-DE-16-121-020 and HB-DE-16-121-021 (same chassis, but different neutralizing agent) from example 6 which had been activated by a straightening iron at 160° C. were compared after restyling using a using a curling iron (BaByliss Pro fashion attitude).

| Formulation no. | Neutralizing Agent | FIG. no. |
|---|---|---|
| HB-DE-16-121-001 | Sodium Hydroxide | 10 |
| HB-DE-16-121-013 | Neutrol TE | 11 |
| HB-DE-16-121-020 | Aminomethyl | 12 |
| | propanol | |
| HB-DE-16-121-021 | Triethanolamine | 13 |

### Example 12

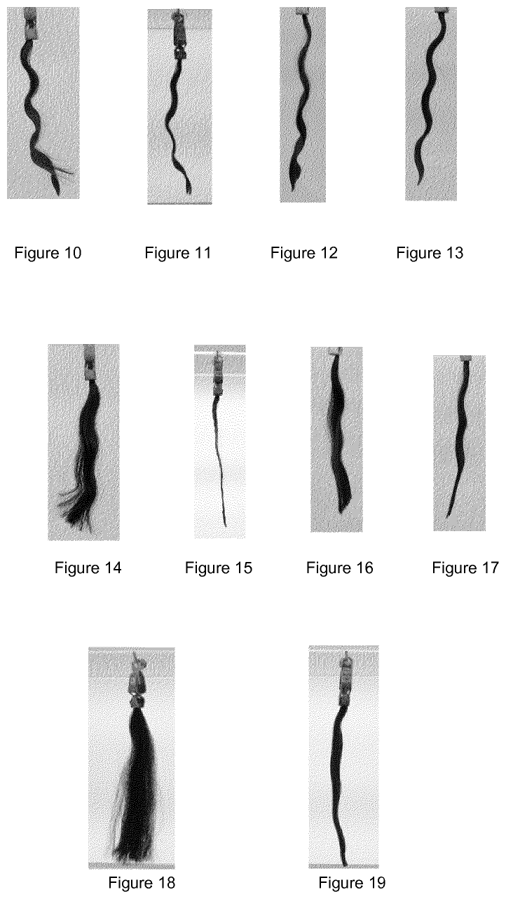

Four hair strands treated with formulation no. HB-DE-16-121-001, HB-DE-16-121-013, HB-DE-16-121-020 and HB-DE-16-121-021 (same chassis, but different neutralizing agent) from example 6 which had been activated by a straightening iron at 160° C. were compared after restyling using a using a crimp iron (Ghd contour professional). FIGS. 14, 15, 16, 17) taken from the side of the hair strands to visualize the styling effect.

| Formulation no. | Neutralizing Agent | FIG. no. |
|---|---|---|
| HB-DE-16-121-001 | Sodium Hydroxide | 14 |
| HB-DE-16-121-013 | Neutrol TE | 15 |
| HB-DE-16-121-020 | Aminomethyl | 16 |
| | propanol | |
| HB-DE-16-121-021 | Triethanolamine | 17 |

### Example 13

The hair strand from example 8 (FIG. 3, right, formulation no. HB-DE-16-121-013) and an only water-treated strand which has also been treated with the curling iron (BaByliss Pro fashion attitude) were put into a climate container for 18 h at 21° C./65% RH.

It can be seen on FIG. 18 that the water-treated strand has spread, the strand treated with formulation no. HB-DE-16-121-013 (FIG. 19) has not spread and shows no frizz.

### Interpretation of the Examples

The examples show that Polyacrylamidomethylpropane Sulfonic Acid can be “heat-activated” (by melting). Hairs treated with this polymer can be easily styled at moderate temperatures using commercially available styling devices like straightening irons, curling irons or crimpers. The styling effect strongly depends on the neutralizing agents used in the formulation. The examples 6, 11 and 12 are showing this surprising effect. The use of organic neutralizing agents, especially Neutrol TE (tetrahydroxypropyl ethylenediamine), enables the restyling of hair strands and results in defined hair styles.

These devices can be run at temperatures from about 160° C. to 230° C. Due to the unique performance of the polymer the preferred application temperature can be from 160° C. to 185° C. Damaging the hair can be avoided in this temperature range and the applied and heat-activated polymer film forms the desired hair styling which is more resistant against mechanical or humidity impacts than heat-treated natural hair.

Example 3 and 4 represent the preferred application procedure. After the product has been applied on wet hair using e.g. a spray, hair cream or conditioner, the hair is dried by using a blow-dryer and a combing device, like brushes or combs. The polymer facilitates the blow-drying/combing procedure and the polymer is homogenously distributed on the hair surface. Afterwards the applied polymer can be “heat-activated” using commercially available styling devices like straightening irons, curling irons or crimpers. If desired it is also possible to restyle the hair, like curling of straightened hair, whilst the polymer film is on the hair. The polymer film can also be easily washed out using standard hair shampoos.

The following formulations have been made and tested. % means % by weight. Rheocare® HSP 1180 from BASF which is used for the following formulations is a product that has a polymer content of between 14 wt.-% AM and 18 wt.-% AM. Thus, the use of 12.5 wt.-% Rheocare® HSP 1180 in a formulation means 2 wt.-% AM polymer in the resulting formulation.

### Example Formulations

Formulation No. HB-DE-16-121-001: a Pumpspray

| | Ingredient | INCI | % |
|---|---|---|---|
| A | Aqua, demin. | Aqua | 74.40 |
| | Ethanol abs. | Alcohol | 10 |
| | Rheocare ® HSP | Polyacrylamidomethylpropane | 12.5 |
| | 1180 | sulfonic acid | |
| | Sodium Hydroxide, | Sodium hydroxide | qs |
| | 10% sol. | | |
| B | Gluadin ® Kera-P LM | Hydrolyzed vegetable protein | 2 |
| | D-Panthenol 75W | Panthenol | 0.50 |
| C | Perfume ,,Cotton | Perfume | 0.30 |
| | Touch“ | | |
| | Eumulgin ® CO 40 | PEG-40 hydrogenated castor oil | 0.30 |
| D | Citric acid, 50% sol. | Citric acid | qs |

Rheocare® HSP 1180 is an aqueous solution containing about 14 to 18% by weight polyacrylamidomethylpropane sulfonic acid.

### Properties:

| | |
|---|---|
| pH value | 4.67 |
| Viscosity (23° C., Brookfield RVT, | 1700 mPa*s |
| Sp4, 10 rpm) | |
| Appearance | slightly yellowish, clear |

### Manufacturing Process:

Dissolve Rheocare HSP® 1180 in a mixture of water and ethanol. Adjust pH to ˜6 with sodium hydroxide and add ingredients of phase B. Solubilize phase C separately and add to the combined phase A+B. If necessary, adjust pH with phase D.

Formulation No. HB-DE-16-121-004: a Serum

| | Ingredient | INCI | % |
|---|---|---|---|
| A | Aqua, demin. | Aqua | 82.20 |
| | Rheocare ® HSP | Polyacrylamidomethylpropane | 12.50 |
| | 1180 | sulfonic acid | |
| | D-Panthenol 75W | Panthenol | 0.50 |
| | Glycerin | Glycerin | 3 |
| | Sodium Benzoate | Sodium benzoate | 0.50 |
| | Sodium Hydroxide, | Sodium hydroxide | qs |
| | 10% sol. | | |
| B | Eumulgin ® CO 40 | PEG-40 hydrogenated castor oil | 0.50 |
| | Copherol ® 1250 C | Tocopheryl acetate | 0.50 |
| | Perfume ,,Cotton | Perfume | 0.30 |
| | Touch“ | | |
| C | Citric acid, 50% sol. | Citric acid | qs |

### Properties:

| | |
|---|---|
| pH value | 4.87 |
| Viscosity (23° C., Brookfield RVT, Sp4, 10 rpm) | 1020 mPa*s |
| Appearance | opaque |

### Manufacturing Process:

Dissolve Rheocare® HSP 1180 in water and adjust pH to ˜4. Add the other ingredients of phase A one by one. Solubilize phase B separately and add to phase A. Adjust pH<5 with phase C if necessary.

Formulation No. HB-DE-16-121-005: a Hair Gel

| | Ingredient | INCI | % |
|---|---|---|---|
| A | Aqua, demin. | Aqua | 69.40 |
| | Ethanol abs. | Alcohol | 10 |
| | Rheocare ® HSP | Polyacrylamidomethylpropane | 12.50 |
| | 1180 | sulfonic acid | |
| | Gluadin ® Kera-P LM | Hydrolyzed vegetable protein | 2 |
| | D-Panthenol 75W | Panthenol | 0.50 |
| | Luviquat ® Supreme | Polyquaternium-68 | 5 |
| | AT1 | | |
| | Sodium Hydroxide, | Sodium hydroxide | qs |
| | 10% sol. | | |
| B | Eumulgin ® CO 40 | PEG-40 hydrogenated castor oil | 0.30 |
| | Perfume ,,Cotton | Perfume | 0.30 |
| | Touch“ | | |
| C | Citric acid, 50% sol. | Citric acid | qs |

### Properties:

| | |
|---|---|
| pH value | 6.41 |
| Viscosity (23° C., Brookfield RVT, | 19200 mPa*s |
| Sp4, 10 rpm) | |
| Appearance | clear, slightly yellowish gel |

### Manufacturing Process:

Dissolve Rheocare® HSP 1180 in a mixture of water and ethanol. Adjust pH to ˜6 with sodium hydroxide. Add the other components of phase A one by one. Solubilize phase B separately and add to phase A. If necessary adjust pH with phase C.

Formulation No. HB-DE-16-121-006: a 2-Phases Pumpspray

| | Ingredient | INCI | % |
|---|---|---|---|
| A | Aqua, demin. | Aqua | 88.95 |
| | Rheocare ® HSP | Polyacrylamidomethylpropane | 6.25 |
| | 1180 | sulfonic acid | |
| | Sodium Hydroxide, | Sodium hydroxide | qs |
| | 10% sol. | | |
| | Plantacare ® 818 UP | Coco-Glucoside | 2 |
| | Plantasil ® Micro | Dicaprylyl Ether, Decyl Glucoside, | 1.50 |
| | | Glyceryl Oleate | |
| | D-Panthenol 75W | Panthenol | 0.50 |
| | Perfume ,,Cotton | Perfume | 0.30 |
| | Touch“ | | |
| B | Euxyl PE 9010 | Phenoxyethanol, Ethylhexylglycerin | 0.50 |
| C | Citric acid, 10% sol. | Citric acid | qs |

### Properties:

| | |
|---|---|
| pH value | 7.10 |
| Viscosity (23° C., Brookfield RVT, | 700 mPa*s |
| Sp4, 10 rpm) | |
| Appearance | white, milky, pearlizing |

### Manufacturing Process:

Dissolve Rheocare® HSP 1180 in water. Adjust pH to ˜6 with sodium hydroxide. Add the other components of phase A. Add phase B. Adjust pH with phase C.

Formulation No. HB-DE-16-121-007: a Pumpspray

| | Ingredient | INCI | % |
|---|---|---|---|
| A | Aqua, demin. | Aqua | 68 |
| | Ethanol abs. | Alcohol | 10 |
| | Rheocare ® HSP | Polyacrylamidomethylpropane | 12.50 |
| | 1180 | Sulfonic acid | |
| | Sodium Hydroxide, | Sodium hydroxide | qs |
| | 10% sol. | | |
| B | Gluadin ® Kera-P LM | Hydrolyzed vegetable protein | 2 |
| | D-Panthenol 75W | Panthenol | 0.50 |
| | Luviquat ® Supreme | Polyquaternium-68 | 5 |
| | AT1 | | |
| | Sodium Chloride | Sodium chloride | 1.40 |
| C | Eumulgin ® CO 40 | PEG-40 hydrogenated castor oil | 0.30 |
| | Perfume ,,Cotton | Perfume | 0.30 |
| | Touch“ | | |
| D | Citric acid, 10% sol. | Citric acid | qs |

### Properties:

| | |
|---|---|
| pH value | 6.11 |
| Viscosity (23° C., Brookfield RVT, | 1060 mPa * s |
| Sp4, 10 rpm) | |
| Appearance | clear, yellowish |

### Manufacturing Process:

Dissolve Rheocare® HSP 1180 in a mixture of water and ethanol. Adjust pH to ˜6 with sodium hydroxide. Add the other components of phase A one by one. Solubilize phase B separately and add to phase A. Adjust pH with phase C. Add phase D.

Formulation No. HB-DE-16-121-008: a Mousse

| | Ingredient | INCI | % |
|---|---|---|---|
| A | Aqua, demin. | Aqua | 87.95 |
| | Rheocare ® HSP | Polyacrylamidomethyl- | 6.25 |
| | 1180 | propane sulfonic acid | |
| | Sodium Hydroxide, | Sodium hydroxide | qs |
| | 10% sol. | | |
| | Plantacare ® 818 UP | Coco-Glucoside | 2 |
| | Plantasil ® Micro | Dicaprylyl Ether, Decyl | 1.50 |
| | | Glucoside, Glyceryl Oleate | |
| | D-Panthenol 75W | Panthenol | 0.50 |
| | Perfume ,,Cotton | Perfume | 0.30 |
| | Touch” | | |
| | Nutrilan ® Milk EP | Hydrolyzed Milk Protein | 1 |
| | Euxyl PE 9010 | Phenoxyethanol, Ethylhexylglycerin | 0.50 |
| B | Citric acid, 10% sol. | Citric acid | qs |

### Properties:

| | |
|---|---|
| pH value | 6.65 |
| Viscosity (23° C., Brookfield RVT, | 440 mPa * s |
| Sp4, 10 rpm) | |
| Appearance | white, milky |

### Manufacturing Process:

Dissolve Rheocare® HSP 1180 in water. Adjust pH to ˜6. Add the other components of phase A. Adjust pH with phase B.

The formulation is filled into an appropriate aerosol container and charged with either one of the following propellants or a mixture in a ratio between 90:10 and 96:4 (ratio solution:propellant):
Propane/Butane 2.7 bar
Propane/Butane 3.5 bar
Propane/Butane 4.5 bar
Isobutane
n-Butane
HFC 152a
   Formulation No. HB-DE-16-121-012: a Pump Mousse

| | Ingredient | INCI | % |
|---|---|---|---|
| A | Aqua, demin. | Aqua | 81.7 |
| | Rheocare ® HSP | Polyacrylamidomethyl- | 12.5 |
| | 1180 | propane sulfonic acid | |
| | AMP Ultra PC 2000 | Aminomethyl propanol | qs |
| | Plantacare ® 818 UP | Coco-Glucoside | 2 |
| | Plantasil ® Micro | Dicaprylyl Ether, Decyl | 1.50 |
| | | Glucoside, Glyceryl Oleate | |
| | D-Panthenol 75W | Panthenol | 0.50 |
| | Perfume ,,Cotton | Perfume | 0.30 |
| | Touch” | | |
| | Nutrilan ® Milk EP | Hydrolyzed Milk Protein | 1 |
| | Sodium Benzoate | Sodium benzoate | 0.50 |
| B | Citric acid, 10% sol. | Citric acid | qs |

### Properties:

| | |
|---|---|
| pH value | 4.84 |
| Viscosity (23° C., Brookfield RVT, Sp4, 10 rpm) | 1080 mPa * s |
| Appearance | clear, slightly yellowish |

### Manufacturing Process:

Dissolve Rheocare® HSP 1180 in water. Adjust pH to ˜4. Add the other components of phase A. Adjust pH to <5 with phase B.

Formulation No. HB-DE-16-121-013: a Pumpspray

| | Ingredient | INCI | % |
|---|---|---|---|
| A | Aqua, demin. | Aqua | 74.40 |
| | Ethanol abs. | Alcohol | 10 |
| | Rheocare ® HSP | Polyacrylamidomethyl- | 12.5 |
| | 1180 | propane sulfonic acid | |
| | Neutrol ® TE | Tetrahydroxypropyl ethylenediamine | qs |
| B | Gluadin ® Kera-P LM | Hydrolyzed vegetable protein | 2 |
| | D-Panthenol 75W | Panthenol | 0.50 |
| C | Perfume ,,Cotton | Perfume | 0.30 |
| | Touch” | | |
| | Eumulgin ® CO 40 | PEG-40 hydrogenated castor oil | 0.30 |
| D | Citric acid, 50% sol. | Citric acid | qs |

### Properties:

| | |
|---|---|
| pH value | 4.85 |
| Viscosity (23° C., Brookfield RVT, Sp4, 10 rpm) | 860 mPa * s |
| Appearance | slightly yellowish, clear |

### Manufacturing Process:

Dissolve Rheocare HSP® 1180 in a mixture of water and ethanol. Adjust pH to ˜6 with Neutrol® TE and add ingredients of phase B. Solubilize phase C separately and add to the combined phase A+B. If necessary adjust pH with phase D.

Formulation No. HB-DE-16-121-018: A Mousse

| | Ingredient | INCI | % |
|---|---|---|---|
| A | Aqua, demin. | Aqua | 87.95 |
| | Rheocare ® HSP | Polyacrylamidomethyl- | 6.25 |
| | 1180 | propane sulfonic acid | |
| | AMP Ultra PC 2000 | Aminomethyl propanol | qs |
| | Plantacare ® 818 UP | Coco-Glucoside | 2 |
| | Plantasil ® Micro | Dicaprylyl Ether, Decyl | 1.50 |
| | Oleate | Glucoside, Glyceryl | |
| | D-Panthenol 75W | Panthenol | 0.50 |
| | Perfume ,,Cotton | Perfume | 0.30 |
| | Touch” | | |
| | Nutrilan ® Milk EP | Hydrolyzed Milk Protein | 1 |
| | Euxyl PE 9010 | Phenoxyethanol, Ethylhexylglycerin | 0.50 |
| B | Citric acid, 10% sol. | Citric acid | qs |

### Properties:

| | |
|---|---|
| pH value | 7.0 |
| Viscosity (23° C., Brookfield RVT, | 480 mPa * s |
| Sp4, 10 rpm) | |
| Appearance | slightly yellowish, turbid |

### Manufacturing Process:

Dissolve Rheocare® HSP 1180 in water. Adjust pH to ˜6. Add the other components of phase A. If necessary adjust pH with phase B.

Formulation No. HB-DE-16-121-019: a Pump Mousse

| | Ingredient | INCI | % |
|---|---|---|---|
| A | Aqua, demin. | Aqua | 81.7 |
| | Rheocare ® HSP | Polyacrylamidomethyl- | 12.5 |
| | 1180 | propane sulfonic acid | |
| | Sodium Hydroxide, | Sodium hydroxide | qs |
| | 10% sol. | | |
| | Plantacare ® 818 UP | Coco-Glucoside | 2 |
| | Plantasil ® Micro | Dicaprylyl Ether, Decyl | 1.50 |
| | Oleate | Glucoside, Glyceryl | |
| | D-Panthenol 75W | Panthenol | 0.50 |
| | Perfume ,,Cotton | Perfume | 0.30 |
| | Touch” | | |
| | Nutrilan ® Milk EP | Hydrolyzed Milk Protein | 1 |
| | Sodium Benzoate | Sodium benzoate | 0.50 |
| B | Citric acid, 10% sol. | Citric acid | qs |

### Properties:

| | |
|---|---|
| pH value | 4.85 |
| Viscosity (23° C., Brookfield RVT, Sp4, 10 rpm) | 860 mPa * s |
| Appearance | clear, slightly yellowish |

### Manufacturing Process:

Dissolve Rheocare® HSP 1180 in water. Adjust pH to ˜4. Add the other components of phase A. Adjust pH to <5 with phase B.

Formulation No. HB-DE-16-121-020: a Pumpspray

| | Ingredient | INCI | % |
|---|---|---|---|
| A | Aqua, demin. | Aqua | 74.40 |
| | Ethanol abs. | Alcohol | 10 |
| | Rheocare ® HSP | Polyacrylamidomethylpropane | 12.5 |
| | 1180 | sulfonic acid | |
| | AMP Ultra PC 2000 | Aminomethyl Propanol | qs |
| | Gluadin ® Kera-P LM | Hydrolyzed vegetable protein | 2 |
| | D-Panthenol 75W | Panthenol | 0.50 |
| C | Perfume ,,Cotton | Perfume | 0.30 |
| | Touch” | | |
| | Eumulgin ® CO 40 | PEG-40 hydrogenated castor oil | 0.30 |
| D | Citric acid, 50% sol. | Citric acid | qs |

### Properties:

| | |
|---|---|
| pH value | 4.76 |
| Viscosity (23° C., Brookfield RVT, Sp4, 10 rpm) | 2520 mPa*s |
| Appearance | slightly yellowish, clear |

### Manufacturing Process:

Dissolve Rheocare HSP® 1180 in a mixture of water and ethanol. Adjust pH to ˜6 with aminomethyl propanol and add ingredients of phase B. Solubilize phase C separately and add to the combined phase A+B. If necessary, adjust pH with phase D.

Formulation No. HB-DE-16-121-021: a Pumpspray

| | Ingredient | INCI | % |
|---|---|---|---|
| A | Aqua, demin. | Aqua | 74.40 |
| | Ethanol abs. | Alcohol | 10 |
| | Rheocare ® HSP | Polyacrylamidomethyl- | 12.5 |
| | 1180 | propane sulfonic acid | |
| | Triethanolamin Care | Triethanolamine | qs |
| | Gluadin ® Kera-P LM | Hydrolyzed vegetable protein | 2 |
| | D-Panthenol 75W | Panthenol | 0.50 |
| C | Perfume ,,Cotton | Perfume | 0.30 |
| | Touch” | | |
| | Eumulgin ® CO 40 | PEG-40 hydrogenated castor oil | 0.30 |
| D | Citric acid, 50% sol. | Citric acid | qs |

### Properties:

| | |
|---|---|
| pH value | 4.72 |
| Viscosity (23° C., Brookfield RVT, Sp4, 10 rpm) | 2040 mPa * s |
| Appearance | slightly yellowish, clear |

### Manufacturing Process:

Dissolve Rheocare HSP® 1180 in a mixture of water and ethanol. Adjust pH to ˜6 with triethanolamine and add ingredients of phase B. Solubilize phase C separately and add to the combined phase A+B. If necessary, adjust pH with phase D.

1. A composition comprising
a polymer, wherein the polymer is water-soluble and has a melting point of at least 145° C. and comprises sulfonic acid groups,
and an organic amine having at least two amino groups,
and optionally further cosmetic ingredients.

2. The composition according to claim 1, wherein the polymer is selected from the group consisting of polyacrylamidomethylpropane sulfonic acid and polystyrene sulfonic acid.

3. The composition according to claim 1, wherein the organic amine having at least two amino groups is selected from the group consisting of ethylendiamine, diethylenetriamine, triethylenediamine, tetraethylenepentamine, and tetrahydroxypropyl ethylenediamine.

4. The composition according to claim 3, wherein the organic amine having at least two amino groups is tetrahydroxypropyl ethylenediamine.

5. The composition according to claim 1, wherein the polymer is present in an amount of from 0.1 to 10% by weight.

6. The composition according to claim 1, wherein the organic amine having at least two amino groups is present in an amount that provides a partial or complete neutralization of the polymer, and wherein the composition has a pH value of 1 to 14.

7. The composition according to claim 1, wherein the composition comprises at least one further polymer, wherein the further polymer is present at a concentration of 0.1 to 10% by weight and is selected from the group consisting of nonionic, anionic, and cationic polymers.

8. The composition according to claim 1, wherein the composition is an aerosol formulation or a non-aerosol formulation, and wherein the formulation is selected from the group consisting of a gel, a mousse, a spray, a serum, and a wax.

9. A process for restyling human hair comprising
applying to the human hair a composition comprising a polymer as defined in claim 1, in an amount of from 0.1 to 10% by weight, and an organic amine in an amount that provides a partial or complete neutralization of the polymer, and optionally further cosmetic ingredients,
thereafter heat-treating the hair, together with the composition on its surface, at a temperature of 145 to 250° C., wherein the hair-style is changed during this heat treatment,
thereafter allowing the hair to cool down to a temperature of below 100° C.,
and thereafter changing the hair style by heat-treating the hair, together with the composition on its surface, at a temperature of 145 to 250° C.

10. The process according to claim 9, wherein the composition comprises a polymer, wherein the polymer is water-soluble and has a melting point of at least 145° C. and comprises sulfonic acid groups,
and an organic amine having at least two amino groups,
and optionally further cosmetic ingredients.

11. The process according to claim 9, wherein the changing of the hair style is carried out with a styling device selected from the group consisting of a straightening iron, curling iron, and a crimper.

12. A composition as defined in claim 1 for use in restyling human hair, wherein the restyling comprises
applying the composition to the human hair,
thereafter heat-treating the hair, together with the composition on its surface, at a temperature of 145 to 250° C., wherein the hair-style is changed during this heat treatment,
thereafter allowing the hair to cool down to a temperature of below 100° C.,
and thereafter changing the hair style by heat-treating the hair, together with the composition on its surface, at a temperature of 145 to 250° C.

13. The composition according to claim 1, wherein the polymer is polyacrylamidomethylpropane sulfonic acid.

14. The composition according to claim 1, wherein the polymer is present in an amount of 0.5 to 2.5% by weight.

15. The composition according to claim 1, wherein the polymer has a melting point of at least 160° C.

16. The composition according to claim 1, wherein the optionally further cosmetic ingredients are selected from the group consisting of solvents, rheology modifiers, preservatives, surfactants, emulsifiers, perfumes, pigments, and coloring agents.

17. The process according to claim 9, wherein the organic amine is selected from the group consisting of monoethanolamine, diethanolamine, triethanolamine, aminomethyl propanol, ethylendiamine, diethylenetriamine, triethylenediamine, tetraethylenepentamine, and tetrahydroxypropyl ethylenediamine.

18. The process according to claim 9, wherein the hair is heat-treated at 150 to 230° C.

19. The process according to claim 9, wherein the hair is heat-treated at 150 to 190° C.

20. The process according to claim 9, wherein the hair is heat-treated at 160 to 185° C.
